(19) United States
(12) Patent Application Publication
(10) Pub. No.: US 2021/0308379 A1
(43) Pub. Date: Oct. 7, 2021

(54) AUTOMATIC INJECTOR FOR STANDARD MEDICAL SYRINGES

(71) Applicants: Sergey Valentinovich TARANOV, Voronezh (RU); Andrey Andreevich ANDREEV, Voronezh (RU)

(72) Inventors: Sergey Valentinovich TARANOV, Voronezh (RU); Andrey Andreevich ANDREEV, Voronezh (RU)

(21) Appl. No.: 17/262,002

(22) PCT Filed: Aug. 12, 2019
(86) PCT No.: PCT/RU2019/050129

§ 371 Date: Jan. 21, 2021

(30) Foreign Application Priority Data
Aug. 9, 2018 (RU) 2018129290

(51) Int. Cl. A61M 5/20; A61M 5/315

(52) U.S. Cl. CPC A61M 5/2033; A61M 2005/2086; A61M 5/3158

(57) ABSTRACT

The device relates to medical technology, and more particularly to devices for intramuscular and subcutaneous injection. The present automatic injector for medical syringes consists of a housing, a holder for a syringe, a piston follower, a syringe lifting mechanism, a firing button, a lifting spring, a spring for inserting the needle, a spring for expressing the injectable fluid, a safety lock and a mechanical dynamic brake member in the form of a damping mechanism. The technical result is that of increasing the operating efficiency of the device and of reducing sensations of pain from rapid drug delivery during an injection.

### FIELD OF THE INVENTION

The invention relates to medical engineering, and particularly to devices for intramuscular and subcutaneous injection.

### BACKGROUND OF THE INVENTION

RU 2649504 discloses an automatic injector for conventional medical syringes. The automatic injector for conventional medical syringes comprises a housing, springs, and a syringe holder. A cocking plate is configured to move downward until a retainer of a syringe lifting mechanism is snapped under the physical strength of a human hand, while a plunger follower is configured to cock in accordance with a filling degree of a syringe, thereby rising to the height of a syringe plunger rod. A gear rack and a plunger follower retainer cause the plunger follower to be fixed at different heights. The syringe is inserted into the syringe holder, and the syringe holder is configured to be released by the action of the human hand on a firing button. By using a main spring interacting with the syringe holder, a syringe needle is inserted to a desired depth into a human body, whereupon the syringe holder, the gear rack, the plunger follower, and the plunger follower retainer are lowered to the lowest position in the housing. The plunger follower retainer is configured to be disengaged from the gear rack of a bar of the syringe lifting mechanism, thereby allowing the downward movement of the gear rack and the plunger follower, which makes it possible to express an injection fluid from the syringe. After the gear rack reaches the retainer of the syringe lifting mechanism, the plunger follower retainer is configured to be released, and to raise, under the action of a lifting spring, the syringe and remove the needle from the injection site. The syringe holder, the plunger follower, and the syringe lifting mechanism are set to longitudinal movement by means of guides in the housing.

The disadvantages of the automatic injector disclosed in RU 2649504 are as follows: its rough operation, very fast drug administration, and slow cocking of the device into its original operating state.

RU 2571231 discloses another automatic injector. The invention relates to medical engineering, and particularly to automatic injectors for administering liquid drug doses. The injector comprises: a tubular casing, a support subassembly, an overlapping initiator cuff, an adjustment spring, four locking means. The support subassembly comprises a tubular support element configured to slide relative to the casing and partially built therein; the support element comprises a syringe with a hollow injection needle, an actuating spring and a plunger used to direct a load of the actuating spring to a syringe retainer. The syringe is fixed together with the support element for their joined axial movement. The overlapping initiator cuff superposes a distal end of the automatic injector and extends at least almost throughout the length of the auto-injector. The adjustment spring surrounds the support element. The first locking means couples a proximal end of the adjustment spring either to the support element to advance it for the purpose of its insertion into the needle, or to the casing to draw the needle back, depending on the relative axial position of the support element and the initiator cuff. The second locking means ensures releasing the actuating spring for injection when the support element is almost found near an injection depth while inserting the needle. The third locking means is configured to couple the casing to the support element for their joined axial movement relative to the initiator cuff; the third locking means enables detaching the casing from the support element when the initiator cuff moves in the proximal direction relative to the casing, thereby releasing the adjustment spring in order to insert the needle. The fourth locking means ensures attaching the distal end of the adjustment spring either to the support element in order to draw the needle back, or to the initiator cuff if otherwise. By using this automatic injector, it is possible to administer drugs of different viscosity, including those of high viscosity, and in different amounts, and prevent drug outflow from the needle. Moreover, this automatic injector may be used by people having dexterity problems.

The disadvantages of the automatic injector disclosed in RU 2571231 are as follows: its great design complexity and difficulty of use.

### SUMMARY

The technical result of the invention consists in increasing the efficiency of device performance, as well as reducing pain from rapid drug administration during injection.

The technical result is achieved by using an automatic injector for medical syringes, which comprises a housing, a syringe holder, a plunger follower, a syringe lifting mechanism, a firing button, a lifting spring, a needle insertion spring, an injection-fluid expression spring, a safety lock and a dynamic brake member in the form of a damper mechanism.

The fact that the main spring is made of the two springs, i.e. the needle insertion spring and the injection-fluid expression spring, makes the device operation smoother and more stable, while the presence of the mechanical dynamic brake member in the form of the damper mechanism ensures slow drug administration from a conventional disposable syringe and causes the device to cock into an operating state in an easy and quick manner.

### BRIEF DESCRIPTION OF THE DRAWINGS

FIG. 1 shows a partial section view of an automatic injector for medical syringes.

FIG. 2 shows an additional partial section view of the automatic injector for medical syringes.

FIG. 3 shows an additional partial section view of the automatic injector for medical syringes.

FIG. 4 shows an additional partial section of the automatic injector for medical syringes.

### DETAILED DESCRIPTION OF THE INVENTION

The device design and operation are explained below.

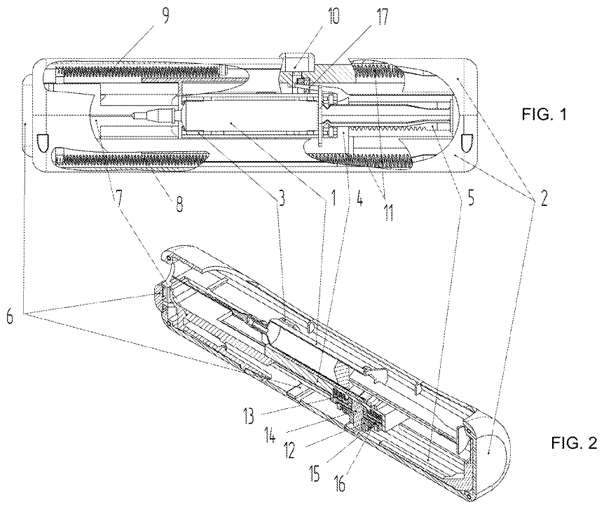

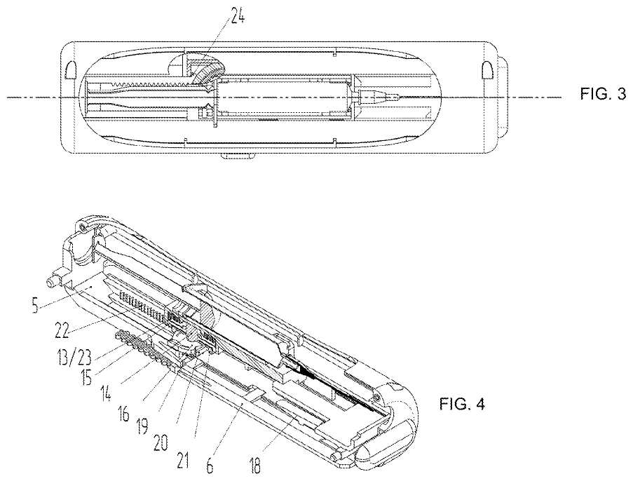

To prepare the device shown in FIG. 1-FIG. 4 for intramuscular injection, it is necessary to fill a medical syringe (1) and put a needle thereon. A transparent cap is removed (not shown in the figures). Injection-fluid expression springs (11) at the amount of 2 pieces are stretched. A lifting mechanism (7) is cocked, and a lifting spring (9) and a needle insertion spring (8) are stretched. When a syringe follower (5) is cocked, it is on a follower retainer (17). When the lifting mechanism (7) is cocked, it is on a lifting mechanism retainer (18). The syringe (1) ready for injection is installed in a cartridge (3) on a cartridge holder (4). Resting on a human body, a safety lock (6) moves and makes it possible to press a button (10). When the button (10) is pressed, the retainer (17) is released from its engagement with a housing (2). The needle insertion spring (8) causes the following elements to move:—the follower (5), the injection-fluid expression springs (11), the cartridge holder (4), the syringe holder cartridge (3) and the entire mechanical dynamic brake member in the form of a damper mechanism (23). This movement is made as far as the cartridge holder (4) stops in the lifting mechanism (7). The syringe needle (1) is inserted to a desired depth into the human body. After that, under the retarding action of the damper mechanism (23), a drug is administered at a slow speed due to the resistance of a damper grease (16) against a damper disk (13). This is due to the interaction of a gear rack (22) with a ratchet gear (12) through a ratchet axis (14) and the damper disk (13) which is closed by a damper cover (15). At the end of injection fluid expression from the syringe (1), the retainer (18) is lifted and disengaged from the housing (2), thereby releasing the lifting mechanism (7) which, under the action of the lifting spring (9), removes the needle from the human body. A cartridge holder retainer (24) is used to prevent the device from being incorrectly cocked into the operating state. For the next injection, it is required to replace the syringe with a new one and repeat all the above operations. A prototype model was manufactured, which exhibited excellent positive results and was much more convenient to use than the prior art analogues.

To allow its sterilization, the prototype model was made of a hygienically approved plastic material that is resistant to antiseptic solutions and resistant to sterilization temperature in autoclaves, and its metal parts were made of stainless steel.

### THE LIST OF REFERENCE NUMERALS

1. conventional disposable syringe

2. housing

3. syringe holder cartridge

4. syringe cartridge holder

5. syringe follower

6. safety lock

7. syringe lifting mechanism

8. needle insertion spring

9. lifting spring

10. firing button

11. injection-fluid expression spring

12. gear ratchet

13. damper disk

14. ratchet axis

15. damper cover

16. damper grease

17. follower retainer

18. retainer

19. gear

20. ratchet

21. ratchet wheel

22. gear rack

23. mechanical dynamic brake member in the form of a damper mechanism

24. cartridge holder retainer

What is claimed is:

1. An automatic injector for conventional medical syringes, comprising: a housing; a syringe holder arranged in the housing; a plunger follower arranged in an upper part of the housing; a syringe lifting mechanism arranged in the housing; a firing button interacting with a need insertion spring and an injection-fluid expression spring; a lifting spring; a safety lock; and a dynamic brake member configured as a damping mechanism retarding the plunger follower.
